SEQUENCE LISTING (8 sequences)

SEQ ID NO: 1 - DNA, 19 bp, Mus musculus
ccatgacgttcctgacgtt

SEQ ID NO: 2 - DNA, 23 bp, Mus musculus
tgccccgcttcctctccaaccaa

SEQ ID NO: 3 - DNA, 20 bp, Mus musculus
tgcctgcagtgcttctaaca

SEQ ID NO: 4 - DNA, 30 bp, Mus musculus
tgacggggtcacccacactgtgcccatcta

SEQ ID NO: 5 - DNA, 30 bp, Mus musculus
ctagaagcatttgcggtggacgatggaggg

SEQ ID NO: 6 - DNA, 19 bp, Mus musculus
tccatgacgttcctgacgt

SEQ ID NO: 7 - DNA, 20 bp, Mus musculus
tgcctgcagtgcttctaaca

SEQ ID NO: 8 - DNA, 23 bp, Mus musculus
tgccccgcttcctctccaaccaa

(19) United States
(12) Patent Application Publication
(10) Pub. No.: US 2009/0220498 A1
(43) Pub. Date: Sep. 3, 2009

(54) Method for Discovering Inhibitors of the Epstein-Barr Virus-Induced Gene 3 (EBI3) and Derivatives Thereof for the Treatment of Metastasizing Tumors and Allergic Asthma

(76) Inventor: Susetta Finotto, Mainz (DE)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/083,706

(22) PCT Filed: Oct. 11, 2006
(86) PCT No.: PCT/EP2006/009833

§ 371 Date: Mar. 17, 2009

(30) Foreign Application Priority Data
Oct. 18, 2005 (DE) 10 2005 049 800.0

(51) Int. Cl. A61K 39/395; C12Q 1/68; C12N 15/11; A61K 48/00; C12Q 1/70; A61K 31/7088; C07K 16/08; A61P 11/06; A61P 35/00

(52) U.S. Cl. 424/133.1; 435/6; 536/24.5; 424/93.21; 435/5; 514/44 A; 530/387.3

(57) ABSTRACT

The present invention relates to a method for identifying inhibitors of the Epstein Barr virus-induced gene 3 (EBI3), a method for producing a pharmaceutical composition, comprising an inhibitor, a respective pharmaceutical composition and a method for treating a metastasizing tumor disease or allergic asthma, comprising the administration of an effective amount of an inhibitor of EBI3.

The present invention relates to a method for identifying inhibitors of the Epstein Barr virus-induced gene 3 (EBI3), a method for producing a pharmaceutical composition, comprising an inhibitor, a respective pharmaceutical composition and a method for treating a metastasizing tumor disease or allergic asthma, comprising the administration of an effective amount of an inhibitor of EBI3.

An infection with Epstein Barr virus (EBV) results in the expression of different antigens, like, for example, the Epstein Barr virus-induced gene (EBI) 3 on infected cells. EBI3 can associate with p28 in order to form IL-27 or can be present as mono-/homodimer. The gene encodes a soluble type 1 cytokine receptor, homolog to the p40 subunit of interleukin 12. Recently, it was found of EBI3 that it associates with a new IL-12 p35-related subunit, referred to as p28, in order to form a non-covalently bridged heterodimeric cytokine (EBI3/p28), referred to as IL-27 [1]. IL-27 (EBI3/p28) is known as an early product of activated antigen-presenting cells which is produced after TLR ligation. It controls the fast clonal expansion of naive, but not of memory CD4⁺ T cells, and is not synergistic with IL-12 in order to elicit the IFN-gamma production via T-bet of naive CD4⁺ T cells [1-3]. The biological function(s) of EBI3 as such or of EBI3/EBI3 homodimers still remain, however, unclear.

Experimental lung melanoma is a disease known for excess Th2 responses and reduced Th1 responses. A possible explanation for the reduced Th1 responses in case of the lung melanoma could be an altered IL-12 production and IL-12 signal transduction [4]. The release of IL-12 (p40/p35) from antigen-presenting cells controls the differentiation of T cells into Th1 cells with up-regulation of IFN-gamma transcription and secretion [6,7]. In addition, IL-12 has a protective role in the lung melanoma, since it is capable of activating cytotoxic lymphocytes, of stimulating natural killer cells, of inducing the production of IFN-gamma and of being synergistic with IL-2.

EBI3 is expressed by monocytes and macrophages similar to IL-12 [10]. In humans, the EBI3 protein is expressed in vivo by dendritic cells (DCs) from lymphoid tissues and in a very high extent by placental syncytiotrophoblasts [10-12]. IL-27 acts in synergy with IL-12 and elicits a fast and clonal expansion of antigen-specific human and murine naive, but not memory CD4⁺ T cells. Its principal function is it to limit the intensity and duration of the unborn and adaptive immune response [12]. The IL-27 receptor is the orphan receptor WSX-1/TCCR associated with gp130 [13]. WSX-1/TCCR deficiency results in an impaired IFN-gamma production and Th1 differentiation and increased susceptibility to infection with intracellular pathogenes [14, 15]. WSX-1 is a new class I cytokine receptor with homology to the IL-12 receptor and is strongly expressed in lymphoid tissue [16]. It was suggested that STAT-1 is activated through the interaction with the tyrosine residue in the cytoplasmatic domain of WSX-1. Furthermore, IL-27 induces the expression of T-bet and IL-12Rbeta2 via WSX-1 in wild type naive CD4⁺ T cells, which indicates that the IL-27/WSX-1 signal transduction is important for the initial determination of Th1 responses [17].

Shrayer et al. [23] describe that IL-12 stimulates the activity of both cytotoxic lymphocytes and natural killer cells and stimulates the production of INF-gamma and can, thus, inhibit the development of different experimental tumors. It was found in mice that the treatment of melanomas with IL-12 (300 ng/day) inhibited the development of primary melanoma tumors in 40% of the mice.

Shrayer et al. [24] further describe that IL-12 can be synergistic with IL-2. Chiyo et al. [25] describe that IL-27 can be composed of p28 and EBI3. The authors investigated whether murine colon 26 colon carcinoma cells retrovirally transduced with the p28-linked EBI3 gene (Colon 26/IL-27) could induce antitumor effects in inoculated mice. Syngene BALB/c mice rejected inoculated Colon 26/IL-27 tumors. However, the authors describe that syngene mice, transduced either with Colon 26/p28 or with Colon 26/EBI3, developed tumors, and that the survival of the mice was identical to the survival of mice inoculated with cells of the primary tumor. Consequently, the authors suggest that only IL-27 expressed in tumors induces a T cell-dependent or independent anti-tumor effect and is a possible therapeutic strategy for cancer.

Allergic asthma can be caused due to environmental allergen exposition in humans reacting allergically. The consequences are attack-like phases of shortage of breath. Frequently, an agonizing permanent cough or allergic permanent cold precedes allergic asthma. Elicitors of allergic asthma seizures are exogenous substances from the environment, like mold spores, house dust, animal skin flakes, animal hair, flower pollen or flour dust. After inhaling, the immune system reacts to the allergens at the bronchia. In patients with allergic asthma hereditary disposition for excess IgE allergen-specific antibody production is often found. Due to an increased secretion of histamine the mucosas swell and excrete stringy mucus. Physical and mental stress as well as viruses can also elicit seizures in case of allergic asthma. Under certain conditions, allergic asthma can be a life-threatening disease. For treatment, usual medicaments (glucocorticoids, often in form of a dosing spray) are used to attenuate the immune system in the area of the airways or to widen the airways.

Hausding et al. [26] describe a role of EBI3 in asthma which is independent of IL-27. EBI3-deficient mice were protected against the development of hyper responses of the airways after inhaling acethylcholin or methacholin and against acidocytosis after allergen sensitizing and aerosolization. These results also show that EBI3 expression per se can elicit immunological responses in the lung (as the experiments in EBI3 transgenic mice show), and, thus, the inhibition of EBI3 allows a respective therapy in case of this indication.

It is, thus, an objective of the present invention to provide an improved treatment of metastatic tumor diseases and of allergic asthma based on the inhibitors of EBI3. It is a further objective of the present invention to identify suitable inhibitors of EBI3 and to make them accessible for such a therapy.

One of the objects of the present invention is solved in a first aspect of this invention by a method for identifying inhibitors of the Epstein Barr virus-induced gene 3 (EBI3). Thereby, the method comprises the steps of a) providing a test system, comprising EBI3 or a biologically active fragment or derivative thereof, b) contacting the test system with one or more compounds, which are assumed to inhibit EBI3, and c) detecting an inhibition of EBI3 by the one or more compounds.

According to the invention a method is preferred, which further comprises the steps of d) identifying the inhibitor of EBI3 or a biologically active fragment or derivative thereof, and, optionally, e) chemically derivatizing the inhibitor selected in step d).

A further object of the present invention is solved in a second aspect of this invention by a method for producing a pharmaceutical composition, comprising a) identifying an inhibitor of EBI3 or a biologically active fragment or derivative thereof as defined above, and b) admixing the inhibitor with a suitable pharmaceutical carrier and/or other suitable pharmaceutical excipients and additives.

A further object of the present invention is solved in a third aspect of this invention by a pharmaceutical composition produced according to the present invention and by an inhibitor of EBI3 identified using a method according to the present invention.

Finally, an even further object of the present invention is solved in a fourth aspect of this invention by a method for treating a metastasizing tumor disease or allergic asthma, comprising the administration of an effective amount of an inhibitor of EBI3 or a biologically active fragment or derivative thereof to a patient.

It was shown of IL-27 that it positively regulates Th1 signal pathways. Furthermore, the inventors have shown earlier that a EBI3 deficiency is associated with a decreased Th2 cytokine production by invariant CD1-restricted T cells and is associated with protection against colitis [18] and asthma. Thus, the inventors wanted to better understand the role of IL-27 and EBI3 in lung melanomas by means of the analysis of EBI3 deficient mice. Similar to previous studies with a Th2-associated colitis [18], the inventors observed that the directed deletion of EBI3 protects against allergic asthma.

The present invention is based on the finding that the Epstein Barr virus (EBV) is a highly antigenic virus, which is caused by the expression of viral antigens, like for example the Epstein Barr virus-induced gene 3 on the surface of infected B cells. The EBI3 gene encodes a soluble type 1 cytokine receptor, homolog to the p40 subunit of interleukin (IL) 12. EBI3 was also found to be associated with a new IL-12 p35-related subunit, referred to as p28, in order to form IL-27, or with the p35 subunit in order to form IL-12. Within the investigations for the present invention the inventors have found a IL-27 independent role of EBI3 in metastatic cancer diseases and allergic asthma and in particular in case of lung melanomas. In fact, EBI3 deficient mice are protected against the development of lung melanomas induced by intravenous injection of B16/F10 cells. Consistently, CD4⁺ T cells from EBI3 deficient mice have an IL-4-dependent defect in the development of T helper cells (Th) 2, because they over-express CTLA-4. Interestingly, not locally derived from the lung but bone marrow-derived, EBI3-deficient dendritc cells (BMDCs) released an increased amount of IL-12 after CpG and LPS stimulation. These results show that a targeting of EBI3 expression and EBI3 function in general and specifically in BMDCs can actuate immunological responses via IL-12 in the lung and, thus, can have important consequences for the design of novel cancer therapies in general and specifically for lung cancer and lung metastasis.

Importantly, the inventors have found that bone marrow-derived dendritic cells (BMDCs), which lack EBI3, can secrete increased amounts of IL-12 and IFN-gamma due to a synergistic TLR ligation. This activating signalling pathway also induces the processed antigen transfer of BMDCs to the lung DCs. Increased IFN-gamma, but not IL-12 of lung DCs, combined with a defect in the IL-4 production was responsible for the final blocking of the development of Th2 cells in the lung. These results show that compared to IL-27 the EBI3 expression per se can cause in BMDCs opposite immunological responses in the lung. These results show that a targeting of EBI3 expression and EBI3 function in tumor and metastatic diseases, like lung melanomas, is advantageous for these diseases in humans.

According to the method of the invention for identifying inhibitors of the Epstein Barr virus-induced gene 3 (EBI3) a test system, comprising EBI3 or a biologically active fragment or derivative thereof, is used—after contacting the test system with one or more compounds, which are assumed to inhibit EBI3—for detecting an inhibition of EBI3 by the one or more compounds.

In a preferred method of the present invention an inhibition of the expression and/or an inhibition of the biological activity of EBI3 or of a biologically active fragment or derivative thereof is detected.

In the first aspect of the present invention, the identification of the role of EBI3 in tumorous diseases provides the possibility of the use of EBI3 as a target for a method for identifying substances that bind to and inhibit EBI3. Methods for routinely performing such screenings are known to the person of skill in the art of pharmaceutics. By using high throughput technologies suitable compound libraries can be screened. These libraries and their screening are known to the skilled artisan and can be readily adapted to the conditions of the present invention without being inventive. For example, U.S. Pat. No. 6,821,737 describes methods and kits for the screening for modulators of transcription factors. The skilled artisan will easily be able to respectively adapt the method described in U.S. Pat. No. 6,821,737 to the present situation.

According to the invention a method is preferred, wherein the test system is selected from purified EBI3, a biologically active fragment or derivative thereof, a cell expressing EBI3, a biologically active fragment or derivative thereof; an in vitro test system; and/or mice, comprising an experimental tumor model. Respective test systems are known to the skilled artisan; these comprise among others the analysis of the expression of gene products to be analysed by means of DNA or RNA analysis, chip-based analysis, RT-PCR, ELISA or other antibody-based detection methods.

The term “biologically active fragment or derivative thereof” within the meaning of the present invention refers to polypeptides, which are functionally related to the EBI3, i.e. which have structural features of this polypeptide. Examples for “derivates” are polypeptides having a sequence homology, in particular a sequence identity, of about 70%, preferably about 80%, in particular about 90%, more particularly about 95% to the polypeptide with the amino acid sequence of EBI3. Included are also additions, inversions, substitutions, deletions, insertions or chemical/physical modifications and/or substitutions or portions of the polypeptide in the range of about 1-60, preferably of about 1-30, in particular of about 1-15, more particularly of about 1-5 amino acids. For example, the first amino acid methionine can be missing without substantially altering the biological function of the polypeptide.

The term “inhibitor” within the meaning of the present invention refers, at one hand, to compounds and/or molecules, that bind to EBI3 and that negatively affect the biological function of the polypeptide, i.e. completely or partially eliminate it. The inhibitor can thereby directly bind to the active site of EBI3 or to a position which has a sterical effect to the active site. Furthermore, the inhibitor can bind in combination with a cofactor, like, for example, a second chemical group, a peptide, protein, or the like. A “inhibitor” within the meaning of the present invention can furthermore eliminate the expression of the gene for EBI3 (e.g. as deletion construct) or can prevent the translation of the EBI3 in the cells. Preferably, the inhibitor of EBI3 or a biologically active fragment or derivative thereof is selected from small molecule chemical compounds, peptides, proteins, nucleic acids, antisense oligonucleotides and antibodies.

More preferably, the inhibitor of EBI3 or a biologically active fragment or derivative thereof is selected from modified p28, modified p35, recombinant antibody fragments and respirable antisense oligonucleotides against the expression of the EBI3 protein. Respective approaches, in particular inhalable oligonucleotides, are also described in detail in the literature [27-33].

A further preferred method of the present invention relates to a method for identifying substances, further comprising a computer-aided structural pre-selection of the one or more compounds, which are assumed to be an inhibitor of EB13 or a biologically active fragment or derivative thereof. By using virtual screening selections can be made of substances, which potentially should inhibit EBI3. These substances are then tested whether they can inhibit the functioning test. A further preferred embodiment of the method of the present invention further comprises a computer-aided structural pre-selection of the one or more compounds, which are assumed to inhibit EBI3. Respective computer-aided methods are known to the skilled artisan.

A further aspect of the present invention relates to a method according to the invention as above, which further comprises the steps of d) identifying the inhibitor of EBI3 or a biologically active fragment or derivative thereof, and, optionally, e) chemically derivatizing the inhibitor selected in step d). If such an inhibitor can be found by means of the test according to the invention, then according to the invention this compound is a lead compound for the further commercial drug development. This compound is then, among other things, used in the following, in particular living test systems and is further developed.

A further preferred embodiment of the method of the present invention further comprises the step of chemically derivatizing the compounds selected as above. As used herein, within the scope of the present invention a “derivative” of a compound identified according to the invention is a derived compound, which is e.g. substituted by different residual groups, as well as mixtures of different of these compounds, which can be processed to a personalized medicament adapted to e.g. the respective disease to be treated and/or to the patient based on diagnostic data or data about the result or progress of the treatment. Within the scope of the present invention a “chemical derivatization” means the method for a respective chemical alteration, such as e.g. the substitution of different residual groups. Preferably, a chemical derivatization is carried out for the purpose of achieving an improved bioavailability or reducing of possible side effects. A “derivative” within the scope of the present invention shall also mean a “precursor” of a substance, which during the course of its administration for treatment is altered by the conditions in the body (e.g. pH in the stomach or the like) in such a way or is metabolized by the body after intake in such a way that the compound according to the invention or its derivatives are formed as active substance.

A further aspect of the present invention then relates to a method for producing a pharmaceutical composition, comprising a) identifying an inhibitor of EBI3 or a biologically active fragment or derivative thereof by means of a method as above, and b) admixing the inhibitor with a suitable pharmaceutical carrier and/or other suitable pharmaceutical excipients and additives, for example a suitable pharmaceutical carrier.

The manufacture of pharmaceutical compositions e.g. in form of medicaments with a content of inhibitor according to the invention or its application in the use according to the invention, respectively, is carried out in the usual way by means of common pharmaceutically technological methods. For this the inhibitors are processed with suitable pharmaceutically acceptable excipients and carriers to dosage forms which are suitable for the different indications and sites of applications.

The medicaments can be produced in such a way that the release rate as desired in each case can be achieved, such as a rapid release and/or a sustained or depot effect, respectively. Thereby, a medicament can be an ointment, gel, patch, emulsion, lotion, foam, cream or mixed-phase or amphiphilic emulsion systems (oil/water-water/oil-mixed phase), liposom, transfersom, paste or powder.

According to the invention the term “excipient” means any, non-toxic, solid or liquid filling, diluting or packaging material, as long as it not unduly disadvantageously reacts with an inhibitor or the patient. Liquid galenic excipients are, for example, water, physiological saline solution, sugar solutions, ethanol and/or oils. Galenic excipients for the manufacture of tablets and capsules can contain, for example, binding agents and filling material.

Furthermore, an inhibitor according to the invention can be used in form of systemically applied medicaments. Including the parenterals, to which belong the injectables and infusions. Injectables are made either in form of ampullae or also as so called ready-to-use injectables, e.g. as ready-to-use syringes or one-way syringes, as well as in injection bottles for several withdrawals. Injectables can be administered in form of subcutanous (s.c.), intramuscular (i.m.), intravenous (i.v.) or intracutanous (i.c.) application. In particular, respectively appropriate injection forms can be made as crystal suspensions, solutions, nanoparticular or colloid-disperse systems, like e.g. hydrosols.

The injectable preparations can further be produced as concentrates, which can be dissolved or dispersed with aqueous isotonic diluents. The infusions can also be prepared in form of isotonic solutions, fat emulsions, liposome preparations, micro emulsions. Like injectables, infusion preparations can be prepared in form of concentrates for dilution. The injectable preparations can also be applied in form of permanent infusions both in stationary therapy and ambulatory therapy, e.g. in form of mini pumps.

The inhibitor according to the present invention can be bound in the parenterals onto microcarrier or nanoparticles, for example onto finely dispersed particles on the basis of poly (meth)acrylates, poly lactates, poly glycolates, poly amino acids or poly ether urethanes. The parenteral preparations can also be modified as depot preparations, e.g. based on the “multiple unit principle” in case that an inhibitor according to the present invention is incorporated in finely dispersed or dispersed, respectively, suspended form or in form of a crystal suspension, or based on the “single unit principle” in case that an inhibitor according to the present invention is included in a dosage form, e.g. a tablet or a stick, which is subsequently implanted. Frequently, these implants or depot medicaments in case of “single unit”- and “multiple unit” dosage forms consist of so called bio-degradable polymers, like e.g. polyester of lactic acid and glycolic acid, poly ether urethanes, poly amino acids, poly (meth)acrylates or poly saccharides.

For the manufacture of parenterals, as excipients and carriers come into consideration: aqua sterilisata, pH-influencing substances, like e.g. organic and inorganic acids and bases as well as their salts, buffering substances for adjusting the pH value, isotonization agents, like e.g. sodium chloride, sodium hydrogen carbonate, glucose and fructose, tensides or surface-active substances, respectively, and emulsifying agents, like e.g. partial fatty acid esters of poly oxy ethylene sorbitan (Tween®) or e.g. fatty acid esters of poly oxy ethylene (Cremophor®), fatty oils, like e.g. peanut oil, soy bean oil and ricinus oil, synthetic fatty acid esters, like e.g. ethyl oleate, isopropyl myristate and neutral oil (Miglyol®), as well as polymeric excipients, like e.g. gelatine, dextrane, poly vinyl pyrrolidone, solubility-increasing additives of organic solvents, like e.g. propylene glycol, ethanol, N,N-dimethylacetamide, propylene glycol, or complexing agents, like e.g. citrates and urea, preservative agents, like e.g. benzoic acid hydroxypropyl ester and methyl ester, benzyl alcohol, antioxidating agents, like e.g. sodium sulfite and stabilizing agents, like e.g. EDTA.

In case of suspensions additives are added: thickening agents for preventing the sedimentation of inhibitors according to the inventions; tensides and peptisation agents in order to ensure that the sediments can be shaken up; or complexing agents, like EDTA. Furthermore, drug complexes can be attained with different polymers, such as with poly ethylene glycols, poly styrol, carboxy methyl cellulose, Pluronics® or poly ethylene glycol sorbitol fatty acid esters. For the manufacture of lyophilisates, scaffolding agents are used, like e.g. mannitol, dextrane, saccharose, human albumin, lactose, PVP or gelatine sorts.

The respectively suitable dosage forms can be manufactured according to formulation instructions and procedures on the basis of pharmaceutical-physical basic principles which are known to the skilled artisan.

A further aspect of the present invention relates then to the respectively produced pharmaceutical composition according to the present invention. This pharmaceutical composition can be characterized in that the compound is available in form of a depot substance or as a precursor together with a suitable pharmaceutically compatible diluting solution or carrier substance.

Preferred is a pharmaceutical composition according to the present invention which contains further chemotherapeutic agents. These chemotherapeutic agents can comprise all chemotherapeutic agents known to the skilled artisan as suitable within a cancer therapy (e.g. Taxol). According to the present invention the above mentioned pharmaceutical composition can be in form of tablets, dragees, capsules, drip solutions, suppositories, injectable or infusion preparations for peroral, rectal or parenteral use. Such dosage forms and their manufacture are known to the skilled person in the art. Particularly preferred is a composition for the administration of a respirable antisense oligonucleotide against the expression of EBI3 protein. Respective approaches, in particular inhalable oligonucleotides, are also described in detail in the literature ([20], [31]-[33]) and can be adapted respectively.

An even further aspect of the present invention relates to a compound, which was identified (selected) by means of one of the methods according to the invention as described above. This compound can be derived from a (commercially available) natural and or synthetic compound library, such compound libraries are well known to the skilled artisan. Preferred is a library with short peptides. In particular preferably, this compound is selected from modified p28, modified p35, recombinant antibody fragments and respirable antisense oligonucleotides (see above).

A further aspect of the present invention relates to an oligonucleotide which specifically hybridizes to the nucleic acid sequence of EBI3. Oligonucleotides are important therapeutics in e.g. gene therapy. The oligonucleotides according to the present invention can be in form of nucleic acids, comprising DNA, dsDNA, RNA, mRNA, siRNA, PNA and/or CNA. The oligonucleotides are preferably “antisense” oligonucleotides. The upper limit for oligonucleotides is determined by the respective practical application, wherein typically a maximal length of 50-200 nucleotides is preferred.

Oligonucleotides are in general rapidly degraded by endonucleases or exonucleases, in particular by DNases and RNases present in the cell. Therefore, it is advantageous to modify nucleic acids in order to protect them against degradation, such that a high concentration of nucleic acid is retained in the cell over a long period of time ([34], [35], WO 95/11910, WO 98/37240; WO 97/29116, Dudycz 1995, Macadam et al. 1998). Typically, such stabilization can be achieved by introducing one or more internucleotide phosphate groups or by introducing one or more non-phosphoric internucleotides.

Suitable modified internucleotides are summarized in Uhlmann and Peymann, 1990 ([36]) (see also [34], [35], WO 95/11910, WO 98/37240; WO 97/29116, Dudycz 1995, Macadam et al. 1998). Modified internucleotide phosphate residues and/or non-phosphor ester bonds in a nucleic acid, which can be employed in one of the uses according to the invention, can contain, for example, methyl phosphonate, phosphoro thioate, phosphor amidate, phosphoro dithioate, phosphate ester, whereas analogues of non-phosphoric internucleotides contain, for example, siloxane bridges, carbonate bridges, carboxy methyl ester, acetamidate bridges and/or thio bridges. It also intended to let this modification improve the storage life of a pharmaceutical composition, which can be employed in one of the uses according to the invention.

With “antisense” oligonucleotides the expression of the respective gene of EBI3 can be reduced in cells both in vivo and in vitro. For the use as an “antisense” oligonucleotide a single stranded DNA or RNA is preferred.

In order to enable the introduction of nucleic acids according to the invention as inhibitors into the cell expressing EBI3 by transfection, transformation or infection, the nucleic acid can be a plasmid, a part of a viral or a non-viral vector. Here, as viral vectors in particular suitable are: retro viruses, Baculoviruses, vaccinia viruses, Adenoviruses, adeno-associates viruses and Herpes viruses. Here, as non-viral vectors in particular suitable are: virosomes, liposomes, cationic lipids, or polylysine-conjugated DNA.

Examples for gene therapeutically effective vectors are virus vectors, such as Adenovirus vectors or retroviral vectors ([37] and [38]).

A further subject of the present invention is an inhibitor in form of a polyclonal or monoclonal antibody or an EBI3-binding fragment thereof, preferably a monoclonal antibody. The term antibody according to the invention means also antibodies produced by means of genetic engineering and optionally modified antibodies or antigen-binding portions thereof, respectively, like e.g. chimeric antibodies, humanized antibodies, multifunctional antibodies, bispecific or oligospecific antibodies, single stranded antibodies, F(ab) oder F(ab)₂ fragments (see e.g. EP-B1-0 368 684, U.S. Pat. No. 4,816,567, U.S. Pat. No. 4,816,397, WO 88/01649, WO 93/06213, WO 98/24884).

A further aspect relates to the application of inhibitors according to the invention as oligonucleotides in gene therapy by means of common transfection systems, such as e.g. liposomes or “particle gun” technologies.

In a preferred embodiment of the method according to the present invention, structurally similar substances (derivatives) can be produced starting from the structures found, which in the context of a “molecular mimicry” specifically bind onto the target EBI3 of the present pathological mechanism.

A further important aspect of the present invention then relates to the use of an inhibitor of EBI3 according to the invention or a biologically active fragment or derivative thereof, as defined above, for the treatment of metastasizing tumor diseases or allergic asthma. Preferred is a use according to the invention, wherein the inhibitor is a pharmaceutical composition, as above, or a compound, as above. Preferred is a use according to the invention, wherein the metastasizing tumor disease is a primary melanoma.

A further important aspect of the present invention relates then to a method for treating a metastasizing tumor disease or allergic asthma, comprising an inhibition of the expression of EBI3 by administering an effective amount of an inhibitor of EBI3 or a biologically active fragment or derivative thereof to a patient. The invention relates to the correlation of the level of the (among others transcriptional) expression of EBI3 with the metastasizing and allergic asthma as well as with the probability of distant metastasizing of, among others, colon carcinomas. Thus, the potential use of this new gene as a therapy target as an intervention target is also given for the tumor therapy for influencing (preventing) of distant metastasizing in case of, among others, lung carcinoma. According to the invention, an influence for the treatment of tumorous diseases and allergic asthma can comprise the application of pharmaceutical composition according to the invention, as stated above. A further particular aspect of the present invention is, thus, a method for the treatment of a tumorous disease, wherein the tumorous disease is lung or colon cancer or allergic asthma.

A further aspect of the present invention relates to an immune therapy with dendritic cells, which are deficient for EBI3 or are knock-out cells for EBI3. Preferably, the EBI3 deficient dendritic cells or dendritic EBI3 knock-out cells are thereby used for the treatment of diseases, in particular metastasizing tumor diseases or allergic asthma. Preferably, the EBI3 deficient dendritic cells or dendritic EBI3 knock-out cells are thereby used for the manufacture of a medicament for the treatment of diseases, in particular metastasizing tumor diseases or allergic asthma. Further preferred is a use according to the invention, wherein the metastasizing tumor disease is a primary melanoma. It is furthermore preferred that a method for the treatment of a metastasizing tumor disease or of allergic asthma comprises the administration of an effective amount of EB13 deficient dendritic cells or dendritic EBI3 knock-out cells to a patient.

In a further embodiment of the present invention the medicament, which is used according to the present invention, is applied via different administration routes, for example, oral, parenteral, subcutanous, intramuscular, intravenous or intracerebral. The preferred route of administration would be parenteral with a daily dose of the compound for an adult of about 0.01-5000 mg, preferably 1-1500 mg per day. Preferred is the medicament with a dosage of between 30 mg/day and 2000 mg/day, preferably between 100 mg/day and 1600 mg/day, most preferably between 300 to 800 mg/day. The suitable dose can be presented as a single dose or as divided doses, in suitable intervals, for example as two, three, four or more sub-doses per day.

Suitable doses can be readily determined by a person of skill in the art by routine experimentation and can be based on factors, such as the concentration of the active ingredient, body weight and age of the patient or other factors related to the patient or the active ingredient.

Pharmaceutical compositions are in general administered in an amount which is effective for the treatment or prophylaxis of a specific condition or conditions. The initial dosage in the human is accompanied by the clinical monitoring of symptoms, the symptoms of the selected condition. In general, the compositions are administered in an amount of active agent of at least about 100 μg/kg body weight. In most cases they are administered in one or more doses in an amount that does not exceed about 20 μg/kg body weight per day. In the most cases the dose of about 100 μg/kg to about 5 mg/kg body weight daily is preferred.

Particular embodiments of the present invention are clarified by the means of the drawings, the sequence listing and the examples, without being limited thereby.

In the sequence listing SEQ ID Nos. 1 to 8 show oligonucleotides which were used in the context of the present invention.

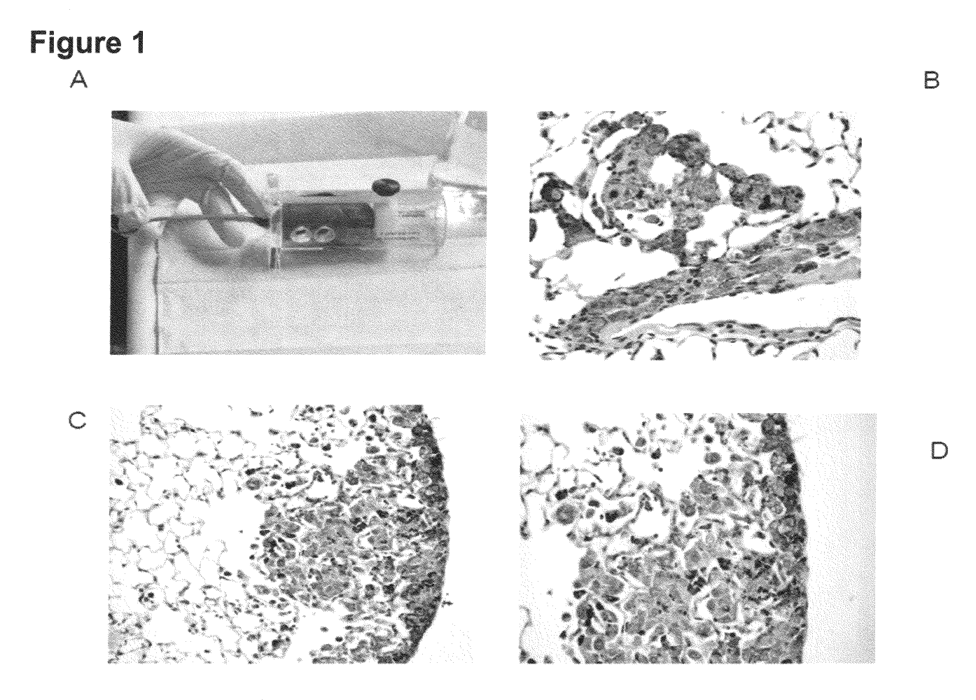

FIG. 1. Intravenous injection of B16/F10 cells induces lung melanomas in a mouse model.

A. 2×10⁵ B16/F10 cells were injected in the tail vein of each mouse.

B. B16/F10 cells can be recognized histologically, because they are loaded with melanine, a brown pigment. In B, in histological sections of the lung B16/F10 cells can be seen, which have left the blood vessels and have entered into the adjacent lymphatic vessels.

In C and D two different magnifications of lung sections can be seen, which show other B16/F10 cells, which migrated to the pleura of the lung, in order to form there macroscopically visible colonies.

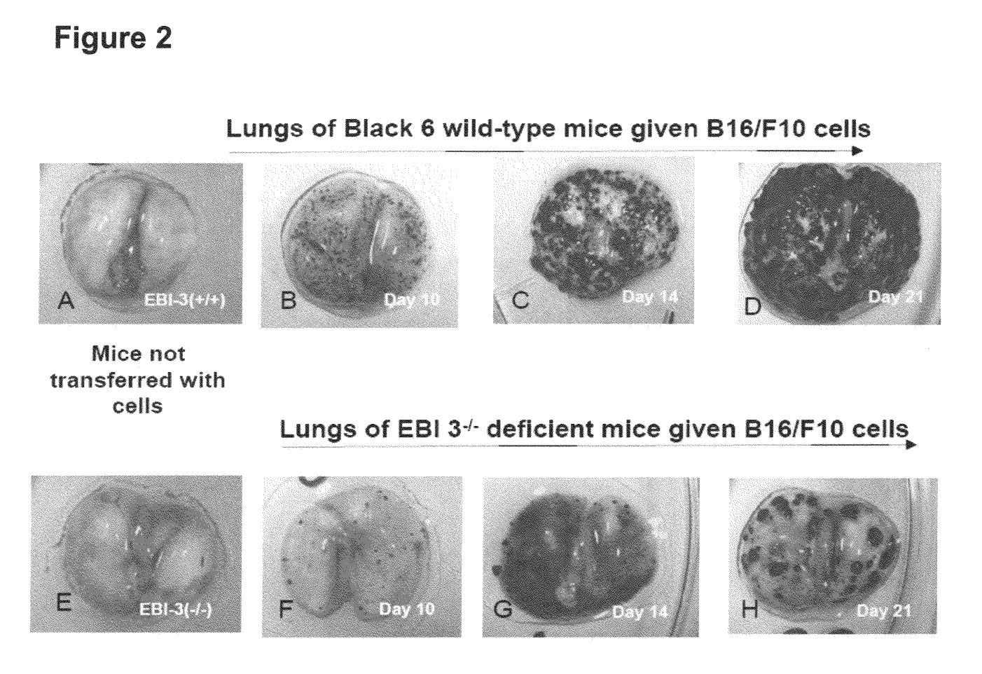

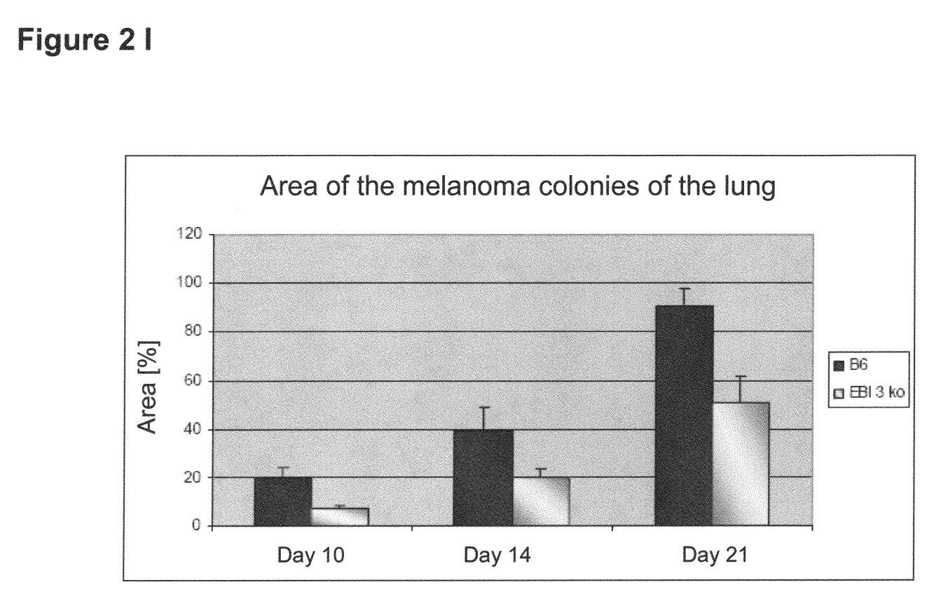

FIG. 2. EBI3 deficient mice are protected against metastasizing melanoma cells in the lung. Intravenous injected B16/F10 cells induce metastatic lung melanomas in C57/BL6-wildtype mice. This tumor develops and progresses in the size of melanotic colonies, starting on day 5 to day 21 after the intravenous B16/F10 cell injection (A-D). Compared to the wildtype litter mates, EBI3 (−/−)-mice are protected against lung melanomas in this model (E-H).

FIG. 2I shows a quantification analysis of the lung colonies 10, 14 and 21 days after intravenous injection in wildtype or EBI3 deficient mice, respectively. EBI3 deficient mice carry a significantly reduced number of colonies/surface, compared to the wildtype litter mates under the same experimental conditions.

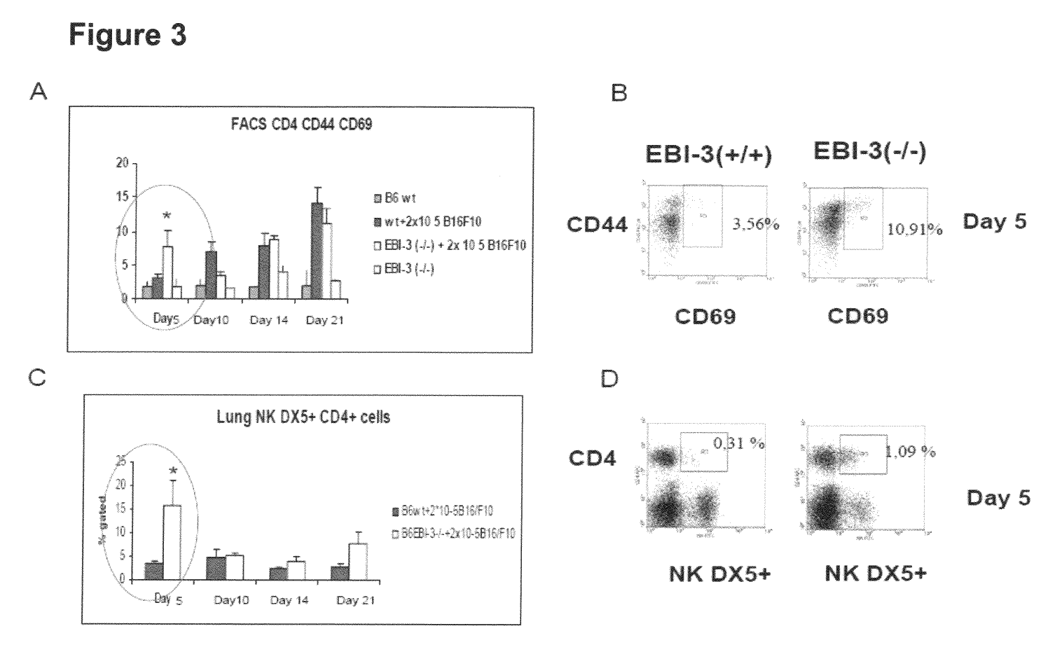

FIG. 3. Increased number of activated memory CD4⁺ T cells and NKDX5⁺ cells in the lung of EBI3-deficient mice 5 days after injection of B16/F10 cells.

Detection of immunological competent cells in lungs of wildtype and EBI3 deficient mice, which at the days shown after intravenous injection of B16/F10 cells were infiltrated. On day 5 a significant increase in CD4⁺CD44⁺CD69⁺ cells were observed in the lung of tumor-carrying EBI3 deficient mice, compared to the wildtype litter mates (A-B). In addition, the number of CD4⁺ NK⁺DX5⁺ increased in the lungs of EBI3 deficient mice, compared to the wildtype litter mates (C-D).

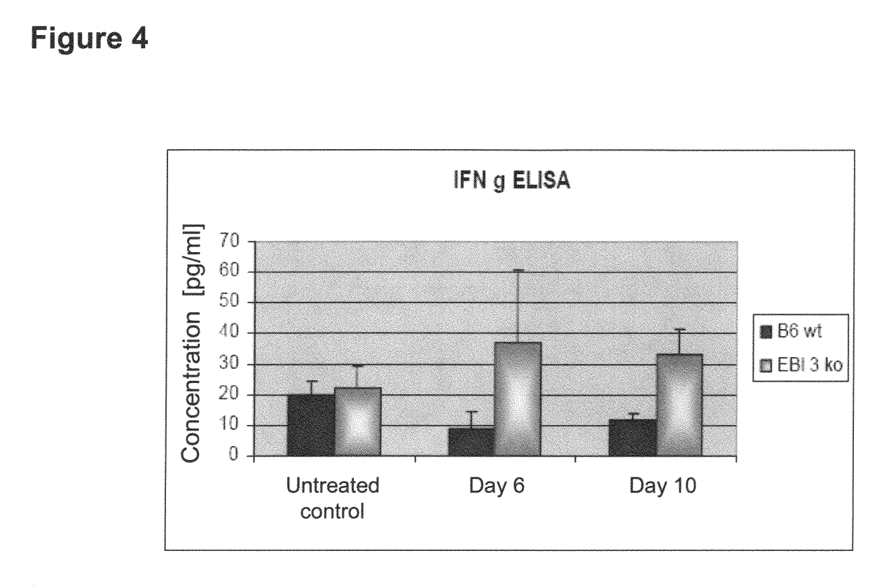

FIG. 4. Increased IFN-gamma release in the airways of melanoma-carrying lungs of EBI3-deficient mice. Bronchoalveolar lavage fluid was obtained from the lung of wildtype and EBI3 deficient tumor-carrying mice. The interferon-gamma analysis was carried out by means of ELISA, as described in Materials and Methods. Compared to the wildtype litter mates, the figure shows that ten days after the injection of B16/F10 cells interferon-gamma is up-regulated in EBI3 deficient mice, which carry tumor 6.

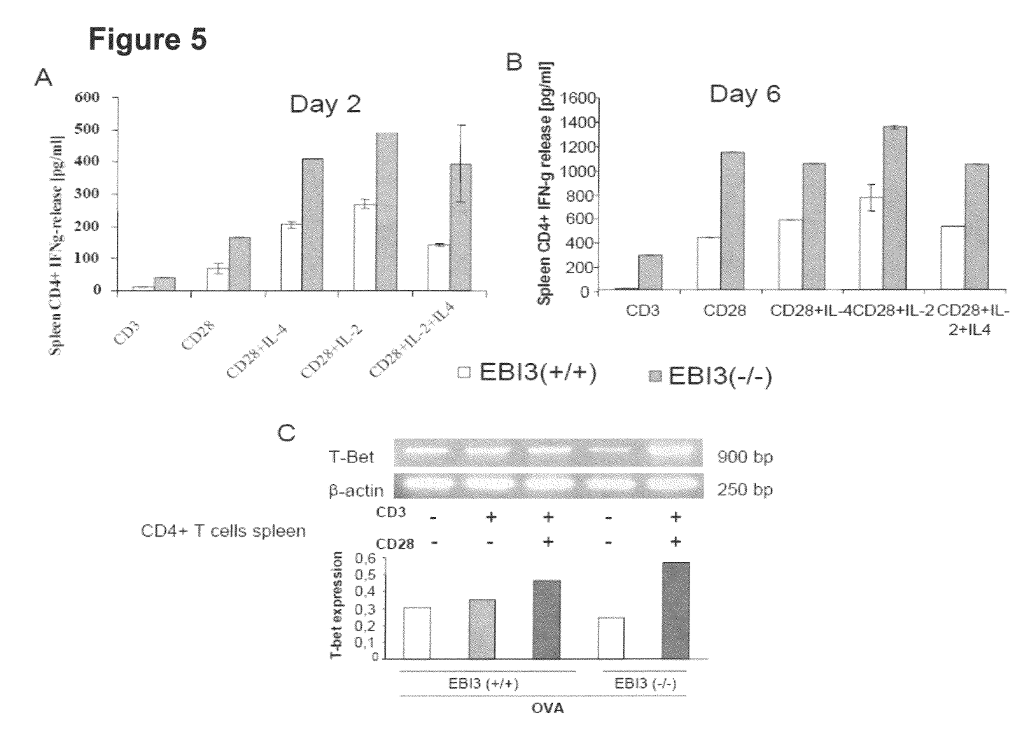

FIG. 5. Co-stimulatory confrontation with anti-CD28 antibodies and IL-4 or IL-2 induces IFN-gamma production by CD4⁺ T cells in EBI3 deficient mice.

CD4⁺ spleen T cells, isolated from EBI3 deficient mice, released in a time-dependent manner an increased amount of IFN-gamma after co-stimulatory confrontation with anti-CD28 antibodies, two (A) and six (B) days after starting the cell culture. Ectopically added IL-4 and IL-2 contributed on day two, but not on day six, to the increased IFN-gamma production by CD4⁺ T cells, which lack EBI3.

C shows RT-PCR analyses of T-bet transcripts from CD4⁺ spleen T cells after indicated confrontation and allergen confrontation with OVA both in wildtype and in EBI3 deficient mice. The highest expression of T-bet was found in CD4⁺ T cells from EBI3 deficient mice after co-stimulatory confrontation with anti-CD28 antibodies, which indicates a T-bet driven INF-gamma production in CD4⁺ T cells, which lack EBI3 after antigen exposure.

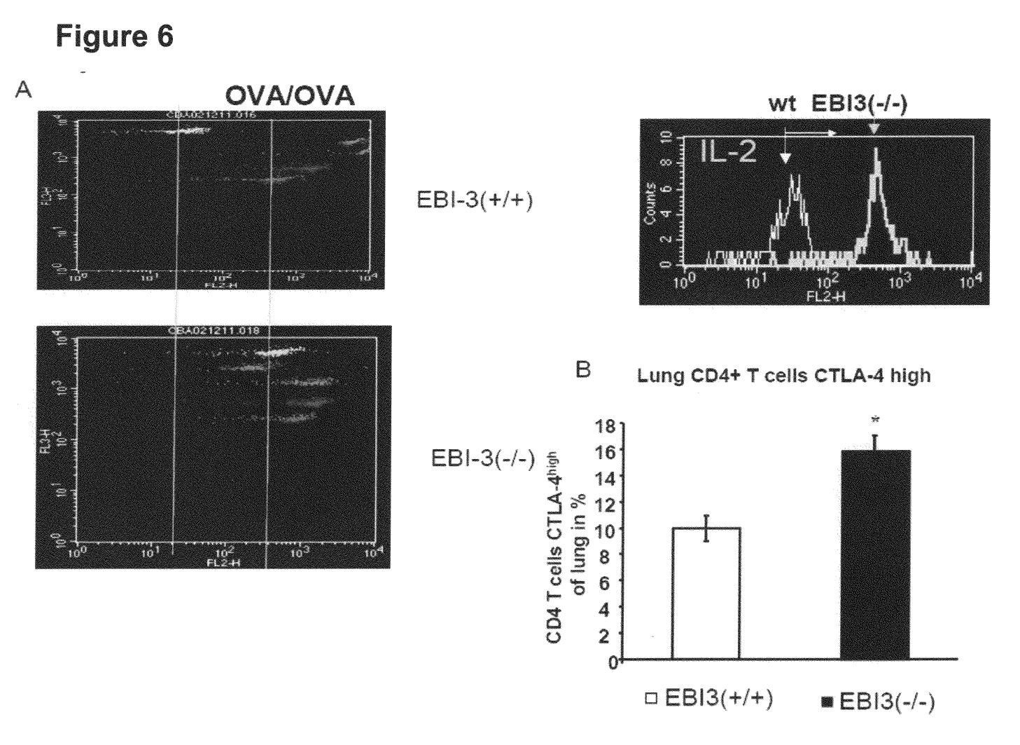

FIG. 6. Increased IL-2 production and CTLA-4 expression by CD4⁺ lung T cells from EBI3 deficient mice.

The targeted deletion of CTLA-4 in mice leads to a reduced production of IL-2 from lymph nodes. In contrast thereto, CD4⁺ T cells isolated from the lung of EBI3 deficient mice increasingly released IL-2, compared to the wildtype litter mates, as CBA-dot blot and histogram analysis show (A).

In addition to that, high CD4⁺ CTLA-4 cells were found in the lung of EBI3 deficient mice, which indicates a negative regulation of the Th2 immune responses in these mice (B).

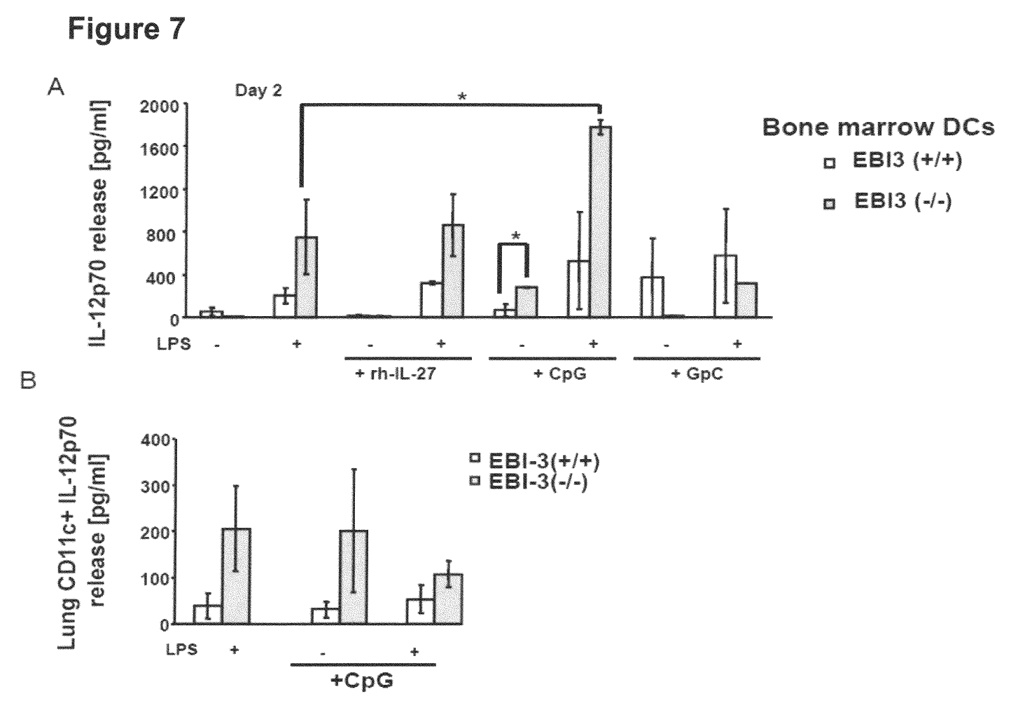

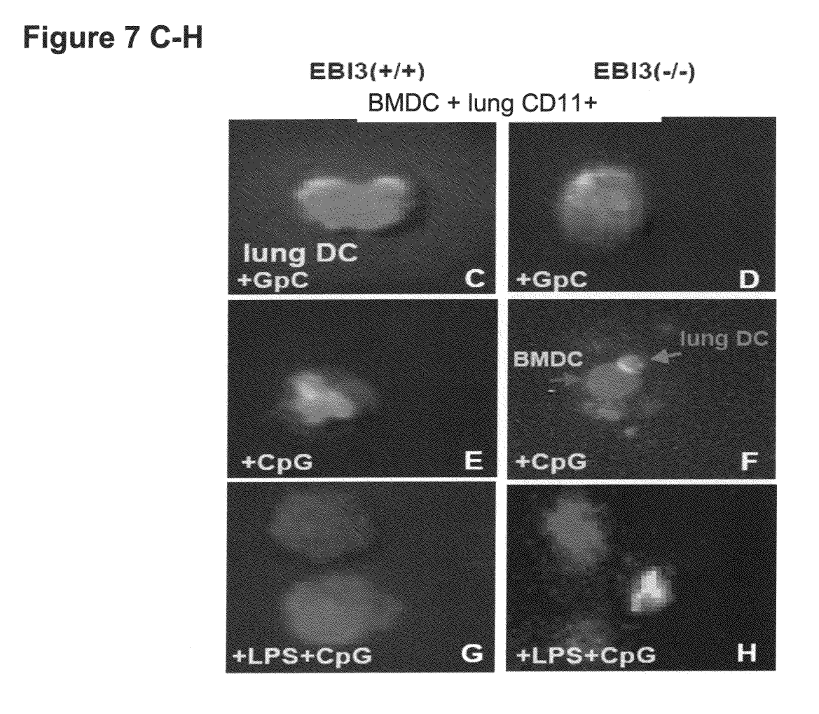

FIG. 7. CpG synergistically amplifies with LPS the IL-12 production in EBI3 deficient bone marrow-derived DCs and causes antigen presentation for resident lung dendritic cells.

BMDCs, which lack EBI3 and which were differentiated in the presence of TNF-alpha and stimulation with TLR-9 and TLR-2/4, released increased amounts of IL-12, compared to DCs, which were isolated from wildtype litter mates (A).

In contrast thereto, lung DCs from EBI3 deficient mice released lower amounts of IL-12, even with co-stimulatory confrontation (B).

Hence, the inventors reason that BMDCs release an increased amount of IL-12 in absence of EB13, that these cells take in the antigen in the periphery and transfer the processed peptide to resident DCs in the lung, which in return control the CD4⁺ T cell responses. In order to emphasize this, the inventors loaded BMDCs from both wildtype and EBI3-deficient mice with Texas Red-labelled ovalbumin and co-cultivated them in presence of CFSE-labelled lung DCs, in order to see, whether the peptide (red) would be transferred to the CFSE-labelled lung DCs. In this case, the green cells would have yellow intra-cytoplasmatic spots (green plus red), which indicates, that OVA is passed between the two DC populations. As FIGS. 7 C-H show, an induced antigen presentation takes place spontaneously with attendance of CpG and LPS or both and induces the IL-12 production.

### EXAMPLES

### Materials and Methods

### Mice, Cytokines and Antibodies

C57/BL6 mice were obtained from Charles River Laboratories. EBI3(−/−)-mice were kept, as previously described, [29, 36, 48]. Purified recombinant mouse-specific IL-4 (10 ng/ml; Peprotech, Rocky Hill, N.J.), anti-CD3 (5 mg/ml; BD PharMingen, Heidelberg, Germany), anti-CD28 (2 mg/ml; BD PharMingen, Heidelberg, Germany), IL-27 (Rand D, Wiesbaden, Germany, LPS (Invivogen, San Diego, Calif.), and CpG (MWG Biotech, Heidelberg, Germany) with the sequence (5′-t(phosphothionated; PTO)CCATGACGTTCCTGACGt(PTO)t(PTO)-3′) (SEQ ID No. 1) or 5′ (Phosphothionate T CC ATG ACG TTC CTG ACG T (Phosphothionate)T (Phosphothionate) 3′ (SEQ ID No. 6)

were used for the in vitro studies.

### Quantification of the Lung Metastases

Metastatic lungs were photographed under the stereo microscope Stemi 200-C with AxioCam MRc. The metastases were labelled at the front site and at the back site of the lung via Axiovision 4.2 from Carl Zeiss Vision GmbH. The labelled areas were summed up and compared to the size of the entire lung. The results are shown as percentage.

### Collection and Analysis of BAL

Bronchoalveolar lavage fluid (BALF) from the right lung was obtained by intratracheal injecting of 0.75 ml saline solution (4×). BALF was collected and an aliquot of cells was stained with Trypan Blue solution for testing the viability using a Neubauer chamber. The samples were spun at 1500 rpm for 5 min and cell pellets were resuspended in 1 ml PBS. Cytospins were carried out by centrifugation at 500 rpm for 5 min. Eosinophilics were determined by staining according to Diff Quick (Dade Behring, Marburg, Germany). The cytospins were analysed with a Zeiss microscope using a 40× objective. The supernatants were frozen and subsequently analyzed by ELISA.

### Isolation and Purification of CD4+ Cells of the Spleen and Lung

Mononuclear cells of the spleen and lung were isolated from freshly obtained samples of healthy C57/BL6 mice (age 6-8 weeks). The lungs were removed, transported on ice in Roswell Park Memorial Institute (RPMI) Medium (Biochrom, Berlin, Germany). Tissue pieces were suspended in Dulbecco's PBS, containing 300 U/ml collagenase type II (Worthington, Lakewood, N.J.) and 0.001% DNase (Roche, Basel, Switzerland). The lung and spleen cells were isolated, as previously described [20]. Briefly, the lung digest was filtered, centrifuged and prior to cell suspension the erythrocytes were removed by hypotonic lysis in ammonium chloride and potassium chloride (ACK) buffer. Lung and spleen CD4⁺ T cells were purified using anti-CD4 beads of Miltenyi (L3T4 beads; Miltenyi, Bergisch-Gladbach, Germany). Lung CD4⁺ T cells were cultivated in RPMI medium in wells coated with anti-CD3 antibodies (5 mg/ml; BD PharMingen, Heidelberg, Germany) in presence of anti-CD28 antibodies (2 mg/ml; BD PharMingen, Heidelberg, Germany) with and without IL-4 (10 ng/ml; Peprotech, Rocky Hill, N.J.) for two days at a density of 10⁶ cells/ml. At this point of time the supernatants were frozen and the cells were incubated for four more days, as described above, in presence or absence of IL-4 (day 6). At this point of time the supernatants were frozen and later analyzed by ELISA for the cytokine production.

Mononuclear cells of the spleen and lung were isolated from freshly obtained samples of healthy C57/BL6 mice (age 6-8 weeks), as previously described (21).

### Differentiation of Bone Marrow-Derived DCs and RNA Extraction

Murine bone marrow-derived DCs (BMDC) were generated, as previously described [22]. Bone marrow cells were isolated from femurs of mice age 6 to 10 weeks and cultivated in serum-free X-Vivo-15 medium (Cambrex, East Rutherford, N.J.), supplemented with 10 ng/ml murine GM-CSF (Peprotech, Rocky Hill, N.J.). Total RNA was isolated using the Trifast reagent (Peqlab, Erlangen, Germany), followed by further purification with the RNeasy MinElute Cleanup Kit (Qiagen, Hilden, Germany) including DNase I digest. 1 to 5 μg RNA were used for the cDNA synthesis with Superscript II (Invitrogen, Heidelberg, Germany). The specific mRNA detection was carried out by PCR analysis with the primers TI#TI previously described [29]. Differentiated BMDCs were then used for the TLR ligation and IL-27-stimulation, as described below, and the supernatants were frozen after 48 hours. Lung dendritic cells were isolated after lung dissociation using anti-CD11c beads of Miltenyi (L3T4 beads; Miltenyi, Bergisch-Gladbach, Germany), as described by the manufacturer.

Isolation of RNA from Lung CD4⁺ T Cells and BMDCs and RT-PCR.

Sorted and cultivated lung CD4⁺ T cells and BMDC cells were immediately frozen after cell culture until RNA isolation, wherein the instructions of the manufacturer were followed (RNeasy Micro Kit; Qiagen, Hilden, Germany), and as previously described (21). 6 μl total RNA from CD4⁺ T cells were reverse transcribed using the RevertAid™ 1^{st} strand cDNA synthesis kits for RT-PCR (M-MuLV reverse transcriptase; MBI-Fermentas GMBH, St. Leon-Rot, Germany). The resulting cDNA was used as template for the PCR. The inventors used the following primers for the T-bet analysis:

(SEQ ID No. 2)

Antisense: 5′ TGC CCC GCT TCC TCT CCA ACC AA 3′,

(SEQ ID No.3)

Sense: 5′ TGC CTG CAG TGC TTC TAA CA 3′
or

(SEQ ID No. 7)

T-bet forward primer: 5′-TGC CTG CAG TGC TTC TAA
CA 3′,

(SEQ ID No. 8)

Reverse Primer: 5′ TGC CCC GCT TCC TCT CCA ACC
AA-3′.

The primers for actin were:

(SEQ ID No. 4)

5′-TGACGGGGTCACCCACACTGTGCCCATCTA-3′
and

(SEQ ID No. 5)

5′-CTAGAAGCATTTGCGGTGGACGATGGAGGG-3′.

The PCR program was as follows: 93° C. 3 min: 32 cycles of 93° C. 30 sec each: 60° C. 30 sec, 72° C. 1 min and final extension 10 min at 72° C. The PCR products were analyzed on 1.5% agarose gels.

### FACS Analysis and Cytometric Bead Array (CBA)

To ensure the purity of the isolated CD4⁺ T cells, routinely 5×10⁵ cells were washed with 1 ml PBS and then incubated for 30 min in 100 μl PBS, containing 5 μg/ml of anti-CD4-AK-FITC (BD PharMingen, Heidelberg, Germany). The cells were washed with 1 ml PBS and then fixed in 1 ml 2% PFA/PBS (Sigma, Deisenhofen, Germany) solution and analyzed. The resulting cell suspensions were measured by FACS Calibur and analyzed using Cell-Quest Pro Version 4.02 (BD PharMingen, Heidelberg, Germany).

CD4⁺ T cells from the lung of OVA-sensitized and confronted mice were incubated overnight in presence of plate-bound anti-CD3 antibodies and soluble anti-CD28 antibodies. The supernatants were analyzed by means of fluorescence cytometry using a cytometric bead array (CBA; mouse Th1/Th2 Kit obtained from BD Bioscience Pharmingen, San Diego, Calif.), wherein the instructions of the manufacturer were followed and as previously described (21). Subsequent to the flow-cytometric acquisition, the sample results were generated in graphic and table formats using the BD CBA analysis software (BD PharMingen, Heidelberg, Germany).

### ELISA.

Murine IL-5 was detected using a specific sandwich ELISA (OptElA™; standard range from 15.6 to 1000 pg/ml; BD PharMingen, Heidelberg, Germany), murine IL-4 was detected using a specific sandwich ELISA (OptElA™; standard range from 7.8 to 500 pg/ml; BD PharMingen, Heidelberg, Germany). IL-13 was detected using a mouse-specific ELISA Kit (Duo set-IL-13; standard range from 40 to 2500 pg/ml, R&D Systems, Wiesbaden, Germany) in the bronchoalveolar lavage fluid. IFN-gamma ELISAs were carried out of BAL and cell supernatants using a sandwich ELISA (OptElA™, standard range from 31.3 to 2000 pg/ml, BD PharMingen, Heidelberg, Germany). IL-12p70 was detected using a mouse-specific ELISA Kit (OptEIA™, standard range from 62.5 to 4000 pg/ml, BD PharMingen, Heidelberg, Germany) in the dendritic cell supernatants.

### Histological Analysis.

For the histological analysis, a significant portion of the lung was prepared and immersed in 10% buffered formalin until it was embedded in paraffin in accordance with standard protocols of the Pathological Department of the University. Histological lung images (40×; image width: each 320 μm) were imported in Photoshop (Adobe Systems Inc. Version 7.0, San Jose, Calif.), as previously described (48).

### Statistical Analysis.

The differences were evaluated using the two-sided Student's t-test for significance (P<0.05) of independent results (Excel, PC). The correlation coefficient was calculated using the statistical analysis of the Excel program. Data are given as means±SD.

### Results

EBI3 Deficient Mice are Protected from Lung Melanomas.

The inventors and others (18) reported about defective Th2 immune responses in EBI3 deficient mice. In this invention the inventors analyzed in an experimental model, whether a EBI3 deficiency would protect against lung melanomas. As shown in FIG. 2 A-D, the intravenous injection of cells of the B16/F10 cell line resulted in the development of lung metastases in a time-dependent manner in wildtype litter mates. In fact lungs of wildtype mice, carrying the tumors, exhibited lung metastases on day 5 to day 21 after the cell injection. At the latest point of time, day 21, about 10-20% of the mice died.

In contrast thereto, the intravenous injection of the B16/F10 cells in EBI3 deficient mice protected these mice against lung metastases, as shown in FIG. 2E-H. The quantitative analysis showed that 10, 14 and 21 days after intravenous injection of B16/F10 cells, EBI3 deficient mice are protected against lung metastases, as is shown by the area occupied with metastatic black colonies (FIG. 21).

### Increased Number of Activated Memory CD4⁺ T Cells and NKDX5⁺ Cells in the Lung of EBI3 Deficient Mice Five Days After the Injection of B16/F10 Lung Melanoma Cells.

In order to understand the immunological mechanisms underlying the tumor escape in EBI3 deficient mice, the inventors dissociated lungs carrying tumors of wildtype and EBI3 deficient mice and carried out a FACS analysis. As shown in FIG. 3A-B, five days after the intravenous injection of 2×10⁵ B16/F10 cells in EBI3 deficient mice an increased number of CD4⁺CD44⁺CD69⁺ cells migrated into the lung, compared to wildtype litter mates. At this point of time also an increased number of CD4⁺ NK⁺DX5⁺ cells from the lung of EB13 deficient mice were observed, compared to wildtype litter mates (FIG. 3C-D), which indicates an increased immune response in the lung of EBI3 deficient mice, compared to wildtype litter mates, after injection of B16/F10 cells.

Increased IFN-Gamma Production in the Lung of EBI3 Deficient Mice Six and Ten Days after Intravenous Injection of B16/F10 Cells.

IFN-gamma is known to have antitumor characteristics and to induce CTL responses. The inventors, thus, analyzed the IFN-gamma release in the lung of mice, which carried melanomas, and found that six and ten days after intravenous B16/F10 cell injection EBI3 deficient mice released an increased amount of IFN-gamma into the airways (bronchoalveolar lavage fluid), compared to wildtype litter mates at the given points in time.

Spleen CD4⁺ T Cells, which Lack EBI3, Released Increased IFN-Gamma after T-Bet Up-Regulation and Co-Stimulation.

EBI3 is produced by dendritic cells after TLR ligation, so far there were no references for a EBI3 release by CD4⁺ T cells. The inventors, thus, analyzed whether the EBI3 deficiency in DCs would affect the cytokine production in CD4⁺ T cells, which were isolated from EBI3 deficient mice. As shown in FIG. 5, CD4⁺ T cells from EBI3 deficient mice released an increased amount of IFN-gamma two and six days after co-stimulation with anti-CD28 antibodies and in presence of either IL-2 or IL-4 or both (FIGS. 5A and 5B, each on day two and day six). The IFN-gamma production was accompanied by the increased expression of the signature TH1 transcription factor T-bet (FIG. 5C).

CD4⁺ T Cells from Lungs of EBI3 Deficient Mice Released an Increased Amount of IL-2 and Expressed Increased Amounts of the Inhibitory Co-Stimulating Molecule CTLA-4.

CTLA-4 is a co-stimulatory molecule, which is associated with the T cell receptor and which negatively regulates the Th2 but not the Th1 differentiation. The inventors, thus, analyzed the CTLA-4 expression in lung CD4⁺ T cells, isolated form EBI3 deficient and wildtype litter mates. As shown in FIG. 6A, CTLA-4 was found to be up-regulated in lung CD4⁺ T cells, isolated form EBI3 deficient mice. Together with this result, the inventors have found that CD4+ T cells, isolated form EB13 deficient mice, released increased amounts of IL-2, as shown by CBA analysis in FIG. 6A.

CpG Synergistically Amplifies with LPS the IL-12 Production by Bone Marrow-Derived DCs (BMDCs), which Lack EBI-3, and Passes the Antigen to Resident Lung Dendritic Cells.

Next the inventors asked whether DCs, isolated from different immunologically relevant sites, would respond differently to the TLR ligation. In this context it was recently described, that BMDCs can be induced by LPS or CpG or by both in a synergistic manner to increasingly produce IL-12 [19]. The inventors, thus, assessed whether EBI3 deficient BMDCs can compensate the defect in IL-27 production by over-production of IL-12 in absence of EBI3. In accordance with previous reports the inventors have found that BMDCs release increased amounts of IL-12 after synergistic LPS and CpG stimuli (FIG. 7A). Interestingly, BMDCs derived from EBI3 deficient mice released significantly more IL-12, compared to the ones isolated from wildtype mice, when both TLR signal pathways (TLR2/4 and 9) were activated (FIG. 7A). Furthermore, lung DCs, isolated from EBI3 deficient mice, did not release as much IL-12 even in presence of toll-like receptor stimulation (FIG. 7B). Hence, the inventors suggest that BMDCs release increased amounts of IL-12 in absence of EBI3, that these cells take in the antigen at the periphery, process it and transfer it to resident DCs in the lung, which in return control the CD4⁺ T cell responses. In order to proof this point, the inventors loaded BMDCs from both wildtype and EB13 deficient mice with Texas Red-labelled OVA and co-cultivated them in presence of CFSE-labelled lung DCs, in order to see, whether the peptide (red) would be passed to the CFSE-labelled lung DCs. In this case, the green cells would have yellow intra-cytoplasmatic spots (green plus red), which would indicate an OVA transfer between the two DC populations. As can be seen in FIGS. 7 C-H, the passed antigen presentation takes place spontaneously and is induced by stimuli, which induce the IL-12 production, namely by CpG and LPS or both.

It was found that the intravenous transfer of in vivo lung DC-EBI-3 (−/−) primed lung CD8+EBI-3 (−/−) T cells were able to heal melanomas in reconstituted wildtype mice, which carried lung melanomas.

It should be understood, that features of the invention, as described and disclosed herein, cannot only be put into practice in the respectively stated combinations, but also in single other ways, without departing from the intended scope of the present invention.

### CITED LITERATURE

1. Pflanz, S., Timans, J. C., Cheng, J., Rosales, R., Kanzler, H., Gilbert, J., Hibbert, L., Churakova, T., Travis, M., Vaisberg, E., Blumenschein, W. M., Mattson, J. D., Wagner, J. L., To, W., Zurawski, S., McClanahan, T. K., Gorman, D. M., Bazan, J. F., de Waal Malefyt, R., Rennick, D., and Kastelein, R. A. (2002). IL-27, a heterodimeric cytokine composed of EBI3 and p28 protein, induces proliferation of naive CD4⁺ T cells. Immunity 16, 779-790.
2. Devergne, O., Hummel, M., Koeppen, H., Le Beau, M. M., Nathanson, E. C., Kieff, E., and Birkenbach, M. (1996). A novel interleukin-12 p40-related protein induced by latent Epstein-Barr virus infection in B lymphocytes. J. Virol. 70, 1143-1153.
3. Devergne, O., Birkenbach, M., and Kieff, E. (1997) Epstein-Barr virus-induziert gene 3 and the p35 subunit of interleukin 12 form a novel heterodimeric hematopoietin. Proc. Natl. Acad. Sci. USA. 94, 12041-12046.
4. Wright, E. D., Christodoulopoulos, P., Frenkiel, S., and Hamid, Q. (1999) Expression of interleukin (IL)-12 (p40) and IL-12 (beta 2) receptors in allergic rhinitis and chronic sinusitis. Clin. Exp. Allergy 29, 1320-1325.
5. Bryan S. A., O'Connor B. J., Matti S., Leckie M. J., Kanabar V., Khan J., Warrington S. J., Renzetti L., Rames A., Bock J. A., Boyce M. J., Hansel T. T., Holgate S. T. Barnes P. J. (2000). Effects of recombinant human interleukin-12 on eosinophils, airway hyper-responsiveness, and the late asthmatic process. Lancet 356, 2149-2153.
6. Rogge, L., Barberis-Maino, L., Biffi, M., Passini, N., Presky, D. H., Gubler, U., and Sinigaglia, F. (1997) Selective expression of an interleukin-12 receptor component by human T helper 1 cells. J. Exp. Med. 185, 825-831.
7. Shevach, E. M., Chang, J. T., and Segal, B. M. (1999) The critical role of IL-12 and the IL-12R beta 2 subunit in the generation of pathogenic autoreactive Th1 cells. Springer Semin. Immunopathol. 21, 249-262. Review.
8. Prescott, S. L., Taylor, A., King, B., Dunstan, J., Upham, J. W., Thornton, C. A., and Holt, P. G. (2003) Neonatal interleukin-12 capacity is associated with variations in allergen-specific immune responses in the neonatal and postnatal periods. Clin. Exp. Allergy 3, 566-572.
9. Busse, W. W., and Lemanske, R. F. Jr. (2001). Asthma. N Engl J. Med. 24, 1643-1644.
10. Devergne, O., Coulomb-L'Hermine, A., Capel, F., Moussa, M., and Capron, F. (2001). Expression of Epstein-Barr virus-induced gene 3, an interleukin-12 p40-related molecule, throughout human pregnancy: involvement of syncytiotrophoblasts and extravillous trophoblasts. Am. J. Pathol. 159, 1763-1776.
11. Hashimoto, S. I., Suzuki, T., Nagai, S., Yamashita, T., Toyoda, N., and Matsushima, K. (2000) Identification of genes specifically expressed in human activated and mature dendritic cells through serial analysis of gene expression. Blood 96, 2206-2214.
12. Hunter C. A. New IL-12-family members: IL-23 and IL-27, cytokines with divergent functions. Nature Reviews-Immunology 5, 521-531.
13. Pflanz, S., Hibbert, L., Mattson, J., Rosales, R., Vaisberg, E., Bazan, J. F., Phillips, J. H., McClanahan, T. K., de Waal Malefyt, R., and Kastelein, R. A. (2004) WSX-1 and glycoprotein 130 constitute a signal-transducing receptor for IL-27. J. Immunol. 172, 2225-2231
14. Villarino, A., Hibbert, L., Lieberman, L., Wilson, E., Mak, T., Yoshida, H., Kastelein, R. A., Saris, C., and Hunter, C. A. (2003) The IL-27R (WSX-1) is required to suppress T cell hyperactivity during infection. Immunity 19, 645-655.
15. Yoshida, H., Hamano, S., Senaldi, G., Covey, T., Faggioni, R., Mu, S., Xia, M., Wakeham, A. C., Nishina, H., Potter, J., Saris, C. J., and Mak, T. W. (2001) WSX-1 is required for the initiation of Th1 responses and resistance to L. major infection. Immunity 15, 569-578.
16. Brombacher, F., Kastelein, R. A., and Alber, G. (2003) Novel IL-12 family members shed light on the orchestration of Th1 responses. Trends Immunol. 24, 207-212. Review.
17. Takeda, A., Hamano, S., Yamanaka, A., Hanada, T., Ishibashi, T., Mak, T. W., Yoshimura, A., and Yoshida, H. (2003) Cutting edge: role of IL-27/WSX-1 signalling for induction of T-bet through activation of STAT1 during initial Th1 commitment. J. Immunol. 170, 4886-4890.
18. Nieuwenhuis, E. E., Neurath, M. F., Corazza, N., Iijima, H., Trgovcic, J., Wirtz, S., Glickman, J., Bailey, D., Yoshida, M., Galle, P. R., Kronenberg, M., Birkenbach, M., Blumberg, R. S. (2002) Disruption of T helper2-immune responses in Epstein-Barr virus induced gene 3-deficient mice. Proc. Natl. Acad. Sci. USA 99, 16951-16956.
19. Napolitani, G., Rinaldi, A., Bertoni, F., Sallusto, F., and Lanzavecchia, A. (2005) Selected Toll-like receptor agonist combinations synergistically trigger a T helper type 1-polarizing program in dendritic cells. Nat. Immunol. 6, 769-776.
20. Finotto, S., Hausding, M., Doganci, A., Maxeiner, J. H., Lehr, H. A., Luft, C., Galle, P. R., Glimcher, L. H. (2005) Asthmatic changes in mice lacking T-bet are mediated by IL-13. Int. Immunol. 17, 993-1007.
21. Doganci, A., Eigenbrod, T., Krug, N., De Sanctis, G. T., Hausding, M., Erpenbeck, V. J., Haddad, E-B., Lehr, H. A., Schmitt, E., Bopp, T., Kallen, K. J., Herz, U., Schmitt, S., Luft, C., Hecht, O., Hohlfeld, J. M., Ito, H., Nishimoto, N., Yoshizaki, K., Kishimoto, T., Rose-John, S., Renz, H., Neurath, M. F., Galle, P. R., and Finotto, S. (2005) The IL-6R alpha chain controls lung CD4+CD25+Treg development and function during allergic airway inflammation in vivo. J. Clin. Invest. 115, 313-325.
22. Wirtz, S., Becker, C., Fantini, M. C., Nieuwenhuis, E. E., Tubbe, I., Galle, P. R., Schild, H. J., Birkenbach, M., Blumberg, R. S., and Neurath, M. F. (2005) EBV-induced gene 3 transcription is induced by TLR signaling in primary dendritic cells via NF-kappa B activation. J. Immunol. 174, 2814-2824.
23. Shrayer D P, Cole B, Hearing V J, Wolf S F, Wanebo H J. (1999) Immunotherapy of Mice with an irradiated melanoma vaccine coupled with interleukin-12. Clin Exp Metastasis. 17(1):63-70.
24. Shrayer D P, Bogaars H, Cole B, Wolf S F, Wanebo H J. (2002) Capacity of murine IL-12 to inhibit the development of primary melanoma tumors and to prevent lung metastases in the melanoma-challenged mice. J Exp Ther Oncol 2(2):93-9.
25. Chiyo M, Shimozato O, Yu L, Kawamura K, Iizasa T, Fujisawa T, Tagawa M. (2005) Expression of IL-27 in murine carcinoma cells produces antitumor effects and induces protective immunity in inoculated host animals. Int J. Cancer. 115(3):437-42.)
26. Hausding M, Wirtz S, Karwot R, Maxeiner J, Lehr H A, Wegmann M, Doganci A, Stememann K, Renz H, Galle P R, Birkenbach M, Neurath M F, Blumberg R S, Finotto S Antigen presentation by Bone Marrow Dendritic Cells (BMDC's) protects EBI3 (−/−) mice from allergic asthma via activation of T-bet, zur Publikation eingereicht.
27. Finotto S. De Sanctis G T, Lehr H A, Herz U, Buerke M, Schipp M, Bartsch B, Atreya R, Schmitt E, Galle P R, Renz H, Neurath M F. Treatment of allergic airway inflammation and hyperresponsiveness by antisense-induced local blockade of GATA-3 expression Exp Med. 2001 Jun. 4; 193(11):1247-60.
28. Finotto S. Buerke M, Lingnau K, Schmitt E, Galle P R, Neurath M F. Local administration of antisense phosphorothioate oligonucleotides to the c-kit ligand, stem cell factor, suppresses airway inflammation and IL-4 production in a murine model of asthma. J Allergy Clin Immunol. 2001 February; 107(2):279-86.
29. Nesterova M, Cho-Chung Y S. Killing the messenger: antisense DNA and siRNA. Curr Drug Targets. 2004 November; 5(8):683-9.
30. Pierce T L, White A R, Tregear G W, Sexton P M. Peptide-oligonucleotide hybrids in antisense therapy. Mini Rev Med Chem. 2005 January; 5(1):41-55.
31. Sandrasagra A, Leonard S A, Tang L, Teng K, Li Y, Ball H A, Mannion J C, Nyce J W. Discovery and Entwicklung of respirable antisense therapeutics for asthma. Antisense Nucleic Acid Drug Dev. 2002 June; 12(3):177-81.
32. Sandrasagra A, Tang L, Leonard S A, Teng K, Li Y, Mannion J C, Nyce J W. RASONs: a novel antisense oligonucleotide therapeutic approach for asthma. Expert Opin Biol Ther. 2001 November; 1(6):979-83.
33. Ball H A, Van Scott M R, Robinson C B. Clin Rev Allergy Immunol. 2004 December; 27(3):207-17 Sense and antisense: therapeutic potential of oligonucleotides and interference RNA in asthma and allergic disorders.
34. Beigelman L, Matulic-Adamic J, Haeberli P, Usman N, Dong B, Silverman R H, Khamnei S, Torrence P F. Nucleic Acids Res. 1995 Oct. 11; 23(19):3989-94: Synthesis and biological activities of a phosphorodithioate analog of 2′,5′-oligoadenylate.
35. Reese C B, Song Q. Nucleic Acids Symp Ser. 1997; (37):1-2: A new approach to oligonucleotide synthesis in solution.
36. Uhlmann E. and Peymann, A. (1990) Antisense oligonucleotides: a new therapeutic principle. Chem. Rev., 90, 544-584.
37. Lindemann D, Patriquin E, Feng S, Mulligan R C. Versatile retrovirus vector systems for regulated gene expression in vitro and in vivo. Mol Med. 1997 July; 3(7):466-76.
38. Springer M L, Chen A S, Kraft P E, Bednarski M, Blau H M. VEGF gene delivery to muscle: potential role for vasculogenesis in adults. Mol Cell. 1998 November; 2(5):549-58.

1. A method for identifying inhibitors of the Epstein Barr virus-induced gene 3 (EBI3), comprising the steps of:
a) providing a test system, comprising EBI3 or a biologically active fragment or derivative thereof,
b) contacting the test system with one or more compounds, which are assumed to inhibit EBI3, and
c) detecting an inhibition of EBI3 by the one or more compounds.

2. The method according to claim 1, wherein the test system is selected from purified EBI3, a biologically active fragment or derivative thereof; a cell expressing EBI3, a biologically active fragment or derivative thereof; an in vitro test system; and/or mice, comprising an experimental tumor model.

3. The method according to claim 1, wherein the inhibition of the expression and/or the inhibition of the biological activity of EBI3 or of a biologically active fragment or derivative thereof is detected.

4. The method according to claim 1, wherein the inhibitor of EBI3 or a biologically active fragment or derivative thereof is selected from small molecule chemical compounds, peptides, proteins, nucleic acids, antisense oligonucleotides and antibodies.

5. The according to claim 4, wherein the inhibitor of EBI3 or a biologically active fragment or derivative thereof is selected from modified p28, modified p35, recombinant antibody fragments and respirable antisense oligonucleotides.

6. The method according to claim 1, further comprising a computer-aided structural pre-selection of the one or more compounds, which are assumed to be an inhibitor of EBI3 or a biologically active fragment or derivative thereof.

7. The method according to claim 1, further comprising the steps of
d) identifying the inhibitor of EBI3 or a biologically active fragment or derivative thereof, and, optionally,
e) chemically derivatizing the inhibitor selected in step d).

8. A method for producing a pharmaceutical composition, comprising the following steps:
a) identifying an inhibitor of EBI3 or a biologically active fragment or derivative thereof according to a method for identifying inhibitors of the Epstein Barr virus-induced gene 3 (EBI3), comprising the steps of:
i) providing a test system, comprising EBI3 or a biologically active fragment or derivative thereof,
ii) contacting the test system with one or more compounds, which are assumed to inhibit EBI3, and
iii) detecting an inhibition of EBI3 by the one or more compounds, and
b) admixing the inhibitor identified in step a) with a suitable pharmaceutical carrier and/or other suitable pharmaceutical excipients and additives.

9. (canceled)

10. A compound identified using a method for identifying inhibitors of the Epstein Barr virus-induced gene 3 (EBI3), comprising the steps of:
a) providing a test system, comprising EBI3 or a biologically active fragment or derivative thereof,
b) contacting the test system with one or more compounds, which are assumed to inhibit EBI3, and
c) detecting an inhibition of EBI3 by the one or more compounds.

11. The compound according to claim 10, selected from modified p28, modified p35, recombinant antibody fragments and respirable antisense oligonucleotides.

12-13. (canceled)

14. A method for the treatment of a metastasizing tumor disease or allergic asthma wherein said method comprises administering, to a patient in need of such treatment, EBI3-deficient dendritic cells, dendritic EBI3-knockout cells, or an inhibitor of EBI3 or a biologically active fragment or derivative thereof.

15. The method according to claim 14, wherein the metastasizing tumor disease is a primary melanoma.

16. (canceled)

17. A pharmaceutical composition comprising a compound of claim 10 and a suitable pharmaceutical carrier and/or other suitable pharmaceutical excipients and additives.

18. The method, according to claim 14, comprising the administration of EBI3-deficient dendritic cells or dendritic EBI3-knockout cells.

19. The method, according to claim 14, comprising the administration of an inhibitor of EBI3 or a biologically active fragment or derivative thereof.

20. The method according to claim 14, wherein the inhibitor is selected from modified p28, modified p35, recombinant antibody fragments and respirable antisense oligonucleotides.
